(19) United States
(12) Patent Application Publication
(10) Pub. No.: US 2026/0151624 A1
(43) Pub. Date: Jun. 4, 2026

(54) METHODS, DEVICES, AND SYSTEMS FOR NON-INVASIVE TREATMENT OF FREQUENT URINATION

(71) Applicant: OHH-MED MEDICAL LTD, Tiberias (IL)

(72) Inventor: Daniel Lischinsky, Ramat Yishai (IL)

(73) Assignee: OHH-MED MEDICAL LTD, Tiberias (IL)

(21) Appl. No.: 19/403,908

(22) Filed: Nov. 30, 2025

Related U.S. Application Data
(60) Provisional application No. 63/726,486, filed on Nov. 30, 2024.

(51) Int. Cl. A61N 1/36; A61N 1/06; A61N 7/00; A61N 7/02

(52) U.S. Cl. CPC A61N 1/36007; A61N 1/06; A61N 1/36034; A61N 7/02; A61N 2007/0021

(57) ABSTRACT

Devices, systems, and methods are claimed for the treatment of male urinary frequency disorders using volumetric tissue heating, e.g., via radiofrequency (RF) or ultrasound (US) energy applied to pelvic floor tissue or penile tissue. In one embodiment, a device for the non-invasive treatment of urinary frequency disorders is disclosed, the device being applicable externally to a perineal region of a male individual, the device comprising: at least one radiofrequency (RF) generator configured to generate RF energy; at least one RF electrode suitable for placement on a target perineal surface; and at least one control circuit connected to the RF generator, wherein the at least one control circuit is operable to cause the RF energy to selectively heat a portion of a urethra, a portion of one or more pelvic floor muscles, a portion of a prostate gland, or a portion of a urinary bladder.

### CROSS-REFERENCE TO RELATED APPLICATIONS

The present application claims priority to U.S. Provisional Ser. No. 63/726,486 , filed on Nov. 30, 2024, the disclosure of which is incorporated herein by reference in its entirety.

The present application is related to U.S. Pat. No. 9,913,981B1 , titled “METHODS AND DEVICES FOR TREATING ERECTILE DYSFUNCTION,” Daniel Lischinsky inventor, currently assigned to Ohh-Med Medical Ltd. and U.S. Patent Application 63/685,279, titled “METHODS, DEVICES, AND SYSTEMS FOR NON-INVASIVE PREVENTION AND TREATMENT OF PREMATURE EJACULATION,” Daniel Lischinsky inventor, which are hereby incorporated by reference to the extent that they are not inconsistent with the instant claims, description, and drawings.

### FIELD OF THE CLAIMED INVENTIONS

The present application relates to methods, devices, and systems using electric currents induced by radio frequency generators or using ultrasound for the neuromodulation of penile and pelvic floor nerves for the treatment of frequent urination disorders; e.g., CPC class code A61N 1/0514.

### BRIEF SUMMARY OF THE INVENTION

Devices, systems, and methods are claimed for the treatment of urinary frequency disorders using volumetric heating, e.g., via radio frequency (RF) or ultrasound (US) energy to target specific areas of the pelvic floor, optionally in combination with warming of penis tissue.

In one embodiment, a method for the non-invasive treatment of frequent urination is disclosed, the method being applicable externally to a perineum of an individual, the method comprising: applying energy non-invasively to a portion of the perineum, wherein the energy is operable to cause volumetric heating of one or more of a portion of a urethra, one or more pelvic floor muscles, a portion of a prostate gland, or a portion of a urinary bladder.

In another embodiment, a method is disclosed for the non-invasive treatment of abnormal urination frequency in a male patient, the method comprising applying energy non-invasively to a skin portion of the male patient, wherein the energy is operable to cause volumetric heating of one or more portions of the ischiocavernosus muscle or one or more portions of the bulbospongiosus muscle.

In yet another embodiment, a device for treating abnormal urination frequency in a male patient is disclosed, the device comprising:
a) at least one energy emitting terminal configured to apply energy non-invasively to a portion of a perineum, wherein the energy is operable to cause volumetric heating of one or more of a portion of a urethra, a portion of one or more pelvic floor muscles, a portion of a prostate gland, or a portion of a urinary bladder;
b) at least one energy emitting terminal configured to apply energy non-invasively to a portion of a penis, wherein the energy is operable to cause volumetric heating of the portion of the penis; and
c) a controller configured to coordinate the operation of the energy emitting terminals of a) and b).

In a further embodiment, a device is disclosed for treating abnormal urination frequency in a male patient, the device comprising:
a) at least one radiofrequency (RF) generator configured to generate RF energy;
b) at least one RF electrode suitable for placement on a target perineal surface;
c) and at least one control circuit connected to the RF generator,
   i. wherein the at least one control circuit is operable to cause the RF energy to selectively heat at least one of a portion of a urethra, a portion of one or more pelvic floor muscles, a portion of a prostate gland, or a portion of a urinary bladder.

The instant disclosure describes technologies for applying energy in a targeted way to the tissues of the pelvic floor or penis, across one or more modalities or domains, including spatial, power, frequency, and time.

In some RF embodiments, one or more electrodes placed at or near the penis may function as a return path or paths for RF electrode(s) on a perineal pad.

### BRIEF DESCRIPTION OF THE DRAWINGS

FIG. 1A depicts a perineal pad embodiment of the instant application, including a plurality of energy emitters.

FIG. 1B depicts a penile pad embodiment of the instant application, including a plurality of return electrodes and energy emitters.

FIG. 2 depicts an inferior-to-superior (bottom-to-top) view of a portion of the male pelvic floor, including musculature.

FIGS. 3A-3C are schematic drawings of a perineal pad embodiment of the instant application, including a 3×3 grid of energy emitters on one side of the pad, shown in FIGS. 3B and 3C.

FIG. 4A depicts the perineal pad embodiment shown in FIG. 3; and FIG. 4B depicts a diagram of the perineal pad embodiment in position under the pelvic floor, including designated heating zones.

FIG. 5 depicts the diagram of the perineal pad embodiment in position under the pelvic floor of FIG. 4B, including designated heating zones and RF electric fields.

FIG. 6 is a schematic drawing of a system for the treatment of urinary frequency disorders, including device components in an example embodiment of the instant application.

FIG. 7 is a flow diagram of a series of operations for the treatment of urinary frequency disorders in example embodiments of the instant application.

### BACKGROUND OF THE INVENTION

In men, increased urinary frequency is a quality of life issue that may increase in severity with age, and which often merits treatment in the form of medication. Efficacy of medications and other treatments is imperfect, however, and there is a need for better treatment options for patients.

Previous work on hyperthermia has been carried out in the vicinity of the perineum for other indications in male patients, however these approaches typically acted one-dimensionally, often with the goal of avoiding pelvic floor tissue heating, and without specific pelvic floor muscle targeting or energy variance across time, space, power, or frequency. See, e.g., U.S. Pat. No. 9,913,981, which relates to the targeting of penile tissue, and intentionally not pelvic floor tissue. These past approaches were of relatively low resolution and relatively imprecise in the use of energy to warm pelvic floor tissues.

The inventor of the technology described herein, in contrast, has discovered that energy can be targeted to specific regions of the pelvic floor, including specific muscles or muscle groups. It has been discovered that this can be beneficial in improving muscle elasticity and urinary frequency problems in male patients. Further, by increasing the resolution and control of energy application to specific pelvic floor tissues, much better control of applied energy can be realized across time, space, power, and frequency domains.

There are several urinary frequency disorders that can affect men, including benign prostatic hyperplasia (BPH), which involves an enlarged prostate that can block the urethra and cause a slow stream of urine. BPH is common, affecting about half of men by age 50. Another urinary frequency disorder is overactive bladder (OAB), which is a health problem that can cause a sudden urge to urinate. OAB can be caused by conditions like Parkinson's disease, multiple sclerosis, and stroke.

There are also various types of incontinence, such as urgency incontinence, which is an involuntary loss of urine that occurs when the bladder muscles contract at the wrong time. It can be caused by BPH, bladder inflammation, bladder stones, or diabetes. Another type is stress urinary incontinence (SUI), which is a condition where urine leaks when physical stress is placed on the bladder, such as when laughing, coughing, or lifting an object.

Other causes of frequent urination in men include bladder cancer, bladder stones, interstitial cystitis (a.k.a., painful bladder syndrome), kidney infection, prostatitis, urethral stricture, neurogenic voiding dysfunction, and urinary tract infection (UTI).

Some treatments for overactive bladder include: bladder retraining, diet modification, Kegel exercises, and biofeedback. Medications for increased urinary frequency include Anticholinergics, which help relax the bladder muscle. Examples include: Oxybutynin (Ditropan XL, Oxytrol, Gelnique), Tolterodine (Detrol, Detrol LA), Darifenacin (Enablex), Solifenacin (Vesicare, Vesicare LS), Trospium (Sanctura XR), and Fesoterodine (Toviaz).

Alpha blockers relax the bladder neck muscles and prostate muscles to make it easier to empty the bladder. Examples include Tamsulosin (Flomax), Alfuzosin (Uroxatral), Silodosin (Rapaflo), and Doxazosin (Cardura).

Mirabegron (Myrbetriq) relaxes the bladder muscle and can increase the amount of urine the bladder can hold.

Botox (Onabotulinumtoxin A) can be injected into the bladder muscle to relax it and increase bladder capacity. It is only recommended for people who can't control symptoms with other medications or behavioral therapies.

### DETAILED DESCRIPTION OF THE INVENTION

Accordingly, the instant application describes improved approaches for the treatment of urinary frequency disorders through the application of energy to heat pelvic floor and other tissues that affect urination, such as the pelvic floor musculature, penile tissue, urethral tissue, prostate gland tissue, and bladder tissue, in a non-invasive fashion.

### Radiofrequency Volumetric Heating Embodiments

Radiofrequency volumetric heating involves the localized application of radiofrequency energy via electrodes, often in a pulsed fashion, for a defined period of time. Effects of applying RF electrical energy to human tissue include localized temperature increases and disruption of nerve signaling. See Weiss RA., “Noninvasive radio frequency for skin tightening and body contouring.” Semin Cutan Med Surg. 2013 Mar; 32(1): 9-17. PMID: 24049924.

Also known as dielectric heating or radiofrequency diathermy, volumetric heating using radiofrequency energy is a technique that involves placing an electrode on the skin near a target tissue and generating electrical current in order to provide heating below the surface of the skin. This non-invasive technique uses radio waves to generate heat in the body's tissues for therapeutic purposes. It has many applications, including pain relief, reducing inflammation, improving blood circulation, accelerating healing, and treating neurological problems.

Diathermy equipment typically operates in the short-wave radio frequency (range 1-100 MHz) or microwave energy (range 434-915 MHz) range.

Various kinds of RF electrodes may be used, including single, bipolar, multipolar, or metal-based flat array electrodes (FAEs).

In some embodiments, a minimally invasive procedure for inducing volumetric heating may be performed, such as Laser Interstitial Thermal Therapy (LITT), RadioFrequency Ablation (RFA), Stereotactic RadioSurgery (SRS), or Radiofrequency Microneedling (RFMN).

In one such embodiment, microneedle RF electrodes may be used to induce an electric field that affects pelvic floor or penile tissue. Examples of microneedle electrodes include microneedle array electrodes (MAEs), insulated or non-insulated radiofrequency microneedling (RFMN), fractional microneedling radiofrequency (FMR), and bipolar microneedle electrode systems. RF microneedling or other minimally invasive approaches are an effective way of delivering current to tissue, especially the superficial nerves of the pelvic floor and penis.

Each of the above RF embodiments may be seen as a stepped-down version of radiofrequency ablation, performed painlessly and without large needles.

The above techniques may be applied to the pelvic floor or penis anatomy in a scheduled manner, as way to induce targeted heating of regions in and around the pelvic floor and bladder area, thereby decreasing the severity of frequent urination symptoms. In some embodiments, treatments may be administered by a physician to empirically determine the appropriate target area(s), RF frequencies, RF power level, and duration of the RF or US to achieve the desired effect. For example, a physician may begin RF treatment at very low power levels, wait for feedback from the patient as to frequent urination symptom status, and increase power level in a subsequent session as needed until an improvement is seen in frequent urination symptoms, e.g., less frequent urination. Monitoring for stability of a clinical effect will determine whether ongoing treatment is necessary.

In some embodiments, treatment may be administered weekly in a doctor's office. In other embodiments, a patient may administer treatment to himself in consultation with a physician as to device placement, power levels, duration of RF energy application, and treatment frequency and duration. Device settings will vary from patient to patient depending on the severity of the patient's urinary frequency problem, the anatomy of the patient, and other factors. For example, power and pulse duration settings may be adjusted depending on the anatomy of the patient, e.g., the size of the patient's pelvic floor region or the amount of adipose tissue in the region of a patient's perineum and pelvic floor.

### Ultrasound Volumetric Heating Embodiments

Ultrasonic volumetric heating delivers low-intensity ultrasound (US) to body tissue, resulting in transient warming of the tissue. See Liu D, Adams MS, Burdette EC, Diederich CJ. “Transurethral high-intensity ultrasound for treatment of stress urinary incontinence (SUI): simulation studies with patient-specific models.” Int J Hyperthermia. 2018 Dec; 34(8): 1236-1247. doi: 10.1080/02656736.2018.1456679. Epub 2018 April 18. PMID: 29566562; PMCID: PMC6136964.

Various ultrasound technologies may be employed in order to effect tissue warming, such as Low Intensity Pulsed Ultrasound (LIPUS).

It has been shown that ultrasound frequencies in a range of from 0.5 to 3 MHz may be used to heat tissues to a depth of at least 5 cm, and ultrasound frequencies and duration may be adjusted depending on the dimensions of the patient's pelvic floor region. The properties of an ultrasound transducer depend upon its diameter and frequency. For example, a small diameter transducer produces a generally small diameter ultrasound beam.

In some embodiments, ultrasound may be administered using one or more transducers fixed on a pad to be placed on the perineum during treatment, and activatable by a controller that can be adjusted by a physician or patient at a user interface.

In the instant application, ultrasound may be used alone as a volumetric heating agent, administered in the region of the perineum and pelvic floor; or US may be used in combination with the RF methods described above.

Combined RF and US therapy may be applied to the perineum alone, or the combination may be applied to the perineum and one or more portions of the penis. Alternatively, RF can be applied to one or more portions of the penis, and US applied to the perineum, or vice versa.

In the following description, various aspects of the present invention are described. For purposes of explanation, specific configurations and details are set forth in order to provide a thorough understanding of the present invention. However, it will also be apparent to one skilled in the art that the present invention as claimed is not limited to the specific details presented herein. Furthermore, well known features may have been omitted or simplified so as to not obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the claimed inventions. In this regard, no attempt is made to show structural details of the claimed inventions in more detail than is necessary for a fundamental understanding of the claimed inventions, the description taken with the drawings making apparent to those skilled in the art how a multitude of forms of the claimed inventions may be embodied in practice.

Before at least one embodiment of the claimed inventions are explained in detail, it is to be understood that the claimed inventions are not limited in its application to the details of construction and the arrangement of the components set forth in the following description or illustrated in the drawings. The claimed inventions are applicable to other embodiments that may be practiced or carried out in various ways as well as to combinations of the disclosed embodiments. Also, it is to be understood that the phraseology and terminology employed herein is for the purpose of description and should not be regarded as limiting.

Methods, systems, and devices for treating urinary frequency disorders are disclosed. Methods are aimed at increasing pelvic floor or penis muscle elasticity and function by delivering energy to specific pelvic floor tissue in the region of the perineum and pelvic floor. Devices may be configured to generate RF energy and to apply the generated RF energy to the perineum and portions of the pelvic floor to thereby elevate a temperature of internal pelvic floor tissue above a predetermined temperature value while maintaining a perineal skin surface below a predetermined temperature threshold. The predetermined temperature value may be set at a level that is effective to alleviate urinary frequency symptoms.

Additionally, as discussed above, ultrasound neuromodulation may also be used either alone or in combination with RF neuromodulation or other urinary frequency disorder therapy modalities to effect optimal results. Advantageously, disclosed embodiments provide preventive, therapeutic, or curative methods and devices for long-term reduction of urinary frequency symptoms, achieved by painless and non-invasive procedures.

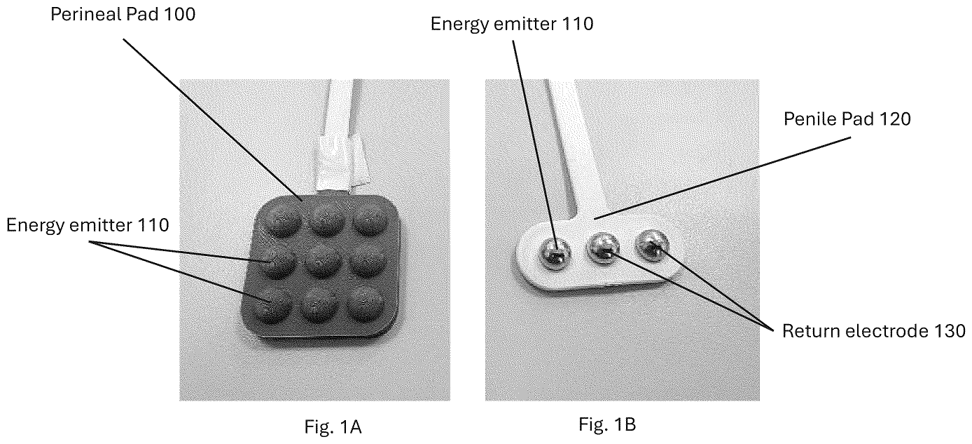

FIG. 1A depicts a perineal pad embodiment of the instant application, including a plurality of energy emitters. Shown are perineal pad 100, with a 3×3 grid of energy emitters 110. In some embodiments, these energy emitters may be RF electrodes or US transducers.

FIG. 1B depicts a penile pad embodiment of the instant application, including a plurality of energy emitters. Shown are penile pad 120, with two return electrodes 130 and an energy emitter 110. In some embodiments, this pad may contain a number of return electrodes to act in conjunction with RF electrodes of a perineal pad 100, or it may include RF electrodes or US transducers for the heating of penile tissue in combination with the heating of pelvic floor or other tissue mediated by energy emitters 100 of perineal pad 100.

In some embodiments, a substantially two dimensional pad configuration is designed for applying RF energy to the perineum. Here, electrodes are affixed within a pad made of, e.g., plastic or fabric, which, when placed in contact with the perineum will generate an RF electric field within pelvic floor tissue. Elevated temperature will then effectuate the desired therapeutic effect on the tissue, which may contribute additively or synergistically to addressing urinary frequency disorder symptoms.

In some embodiments, a sleeve or cuff may be employed to provide support for electrodes. The sleeve or cuff may fit snugly around the lower torso or penis in order to provide contact between electrodes housed therein and the skin (or any thin film between the electrode(s) and the skin). The sleeve or cuff may comprise an elastic material to facilitate fitting and removal. In one embodiment, the electrodes or transducers may be in the form of a bar that can be positioned via the sleeve or other support longitudinally along the perineum or penis, at an angle, or in another position that facilitates the appropriate delivery of energy to the target tissue.

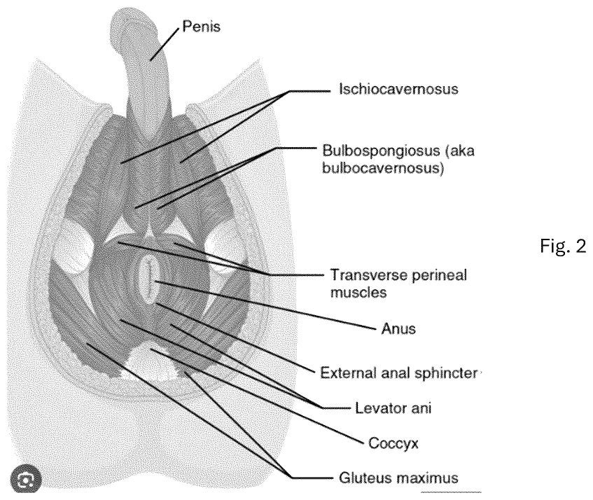

FIG. 2 depicts an inferior-to-superior (bottom-to-top) view of a portion of the male pelvic floor, including some of the musculature.

In males, the pelvic floor comprises four layers moving inferiorly to superiorly from the external perineum:

1^{st} layer: the most inferior tissue is the skin, then the subcutaneous fat, then the fascia of Colles, and finally the superficial perineal fascia.

2^{nd} layer: next is the superficial perineal pouch (bounded superiorly by the perineal membrane and inferiorly by the superficial perineal fascia), including: the superficial transverse perineal muscles (left and right), whose origin is on the tendinous fibers of the inner and forepart of the ischial tuberosity; insertion runs medially to the central tendinous point of the perineum (perineal body); innervation is by the deep perineal nerve; action is to constrict the urethra and maintain urinary continence.

The second layer also includes the bulbospongiosus muscles (left and right; aka bulbocavernosus), whose origin is on the perineal body and the median penile raphe; insertion is on the perineal membrane, the dorsal aspect of the corpus spongiosum and the corpora cavernosa, and the fascia of the bulb of the penis; innervation is by the deep branch of the perineal nerve; action is to compress the bulb of the penis during urination and ejaculation, assists with erection of the penis (with ischiocavernosus), and supports the perineal body.

The second layer also includes the ischiocavernosus muscles (left and right), whose origin is on the ischial tuberosity and the ischial ramus; insertion is on the crus of the penis; innervation is by the deep branch of the perineal nerve; action is to maintain blood flow to the penis. The perineal body and external anal sphincter are located at the posterior of this layer.

3^{rd} layer: next, past the perineal membrane is the deep perineal pouch, which contains part of the urethra, the bulbourethral glands, and the deep transverse perineal muscles (left and right), whose origin is on the inferior rami of the ischium; insertion is on the contralateral deep transverse perineal muscle; innervation is by the deep perineal nerve; action is to constrict the urethra to maintain urinary continence.

4^{th} layer: The fourth layer includes the levator ani muscle group, a.k.a. the pelvic diaphragm, which assists with urinary continence as well as helping to control defecation. The fourth layer also includes the iliococcygeus, the pubococcygeus, the puborectalis, and the ischiococcygeus (a.k.a. the coccygeus; not part of the Levator ani group).

These four layers of pelvic floor tissue are among the target tissues of the instant application.

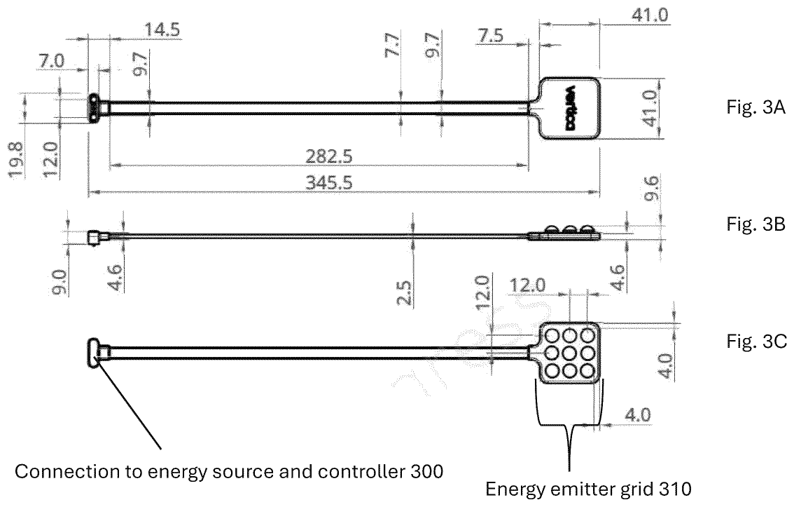

FIGS. 3A-C show schematic drawings of a perineal pad embodiment of the instant application, including a 3×3 grid of energy emitters on one side of the pad. FIG. 3A shows the back of the pad, FIG. 3B shows a side view of the pad, including three rows of raised energy emitters, and FIG. 3C shows a front view of the pad, with energy emitter grid 310. Each of the three figures includes connection to energy source and controller 300.

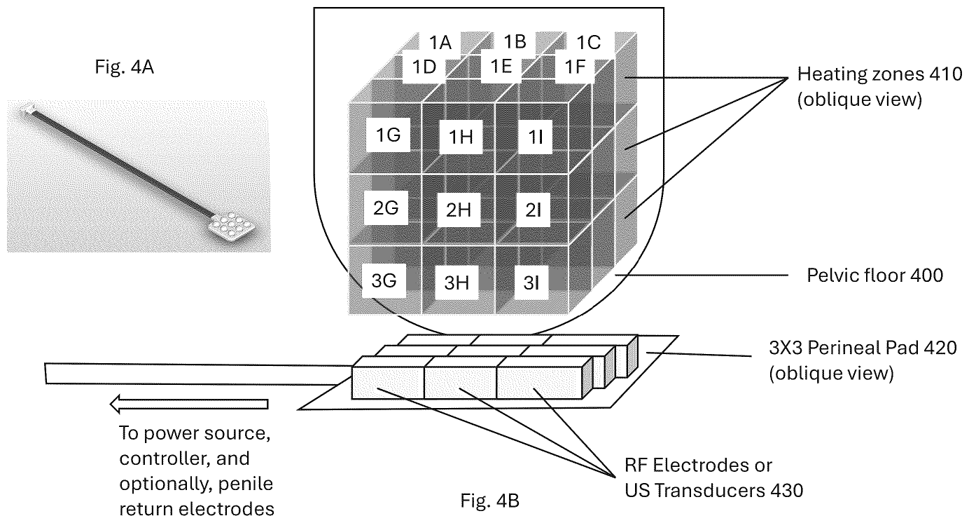

FIG. 4A shows a drawing of a perineal pad embodiment of the instant application, and FIG. 4B shows a diagram of the perineal pad embodiment in position under the pelvic floor, including designated heating zones. As shown in FIG. 4B, RF Electrodes or US Transducers 430 are present in a 3×3 grid on 3×3 perineal pad 420, adjacent to pelvic floor 400. Heating zones 410 represent various locations within the anatomy of the pelvic floor, as discussed above. For example, heating zones 1G, 1H, and 1I are deeper (or more superior) relative to heating zones 3G, 3H, and 3I.

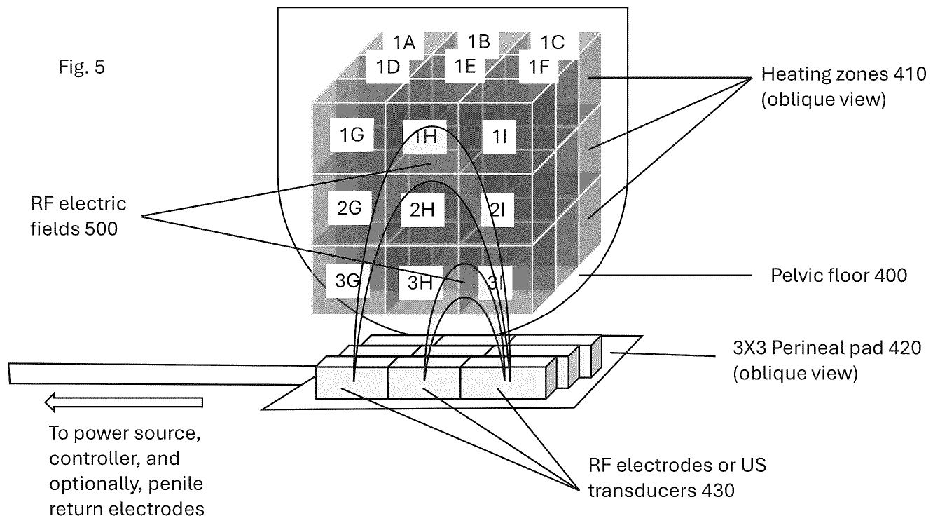

FIG. 5 shows the FIG. 4B diagram with the addition of RF electric fields 500, which are shown penetrating into various heating zones 410.

### Energy Application Across Time, Space, Frequency, and Power Domains

Power levels of RF (or ultrasound), duration of treatment, energy frequency, and the target tissue (anatomically speaking) may be varied depending on the desired effect on the individual. A typical radiofrequency power setting is 15 W. In some embodiments, waves of energy may be applied across anatomical zones in order to heat muscles and other tissue in various ways. For example, application of energy to posterior zones during a first pulse may be followed by application of energy to anterior zones during a second pulse to simulate a wave of heating in the posterior to anterior direction.

Similarly, power levels may be modulated in various zones over time to effectuate alternating high and low temperature periods of therapy, in the same or different spatial domains.

Likewise, various tissue types such as specific muscle groups may be heated in sequence or simultaneously. For example, the left and right bulbospongiosus muscles may be alternately heated by applying energy to the appropriate heating zone at the desired time, for the desired duration of effect.

Power and frequency may be modulated to achieve heating at desired depths according to tissue density, tissue depth, tissue responsiveness (e.g., speed of heating), and initial temperatures. In a preferred embodiment, tissue may be heated using RF or US at a depth of 0.5 to 15 mm beneath the surface of the skin. Alternatively, greater target depths may be heated, e.g., up to 5 cm or more. With greater depths, temperature gradients may be seen across anatomical regions such as muscle groups, which may in some cases be beneficial.

In some embodiments, an RF generator may be configured to generate RF energy at a frequency ranging between 100 KHz-40 MHz. In other embodiments, an RF generator may be configured to generate RF energy at power ranging between 1-100 W. In some embodiments, an RF generator may be configured to generate RF energy at power ranging between 15-25 W. In yet other embodiments, the power of RF energy generated by an RF generator may be determined based on, for example, a geometry of the RF electrodes or RF electrode pairs, such as relative distance between electrodes, or RF power may be based on tissue parameters (e.g., impedance, geometry, size, etc.) of a specific patient undergoing the treatment. In a preferred embodiment, RF frequencies will be at or about 1 MHz, and US frequencies will be at or about 800 kHz.

In some embodiments, RF pulse duration may range from 1 millisecond to 3 seconds. In a preferred embodiment, the pulse duration will be from 1 to 50 milliseconds. In some embodiments, a pulse or a series of pulses may be delivered over the course of up to 15 minutes. Short pulses or series of pulses should not be expected to raise tissue temperature significantly, however longer pulses may increase localized tissue temperatures to between 37 and 96 degrees Celsius. Temperature sensing as described herein will sense skin surface temperature to make sure that skin surface temperatures do not exceed 43 degrees Celsius, to avoid skin burns.

RF generators may include control circuitry connected to each RF electrode or each RF electrode pair. Such control circuitry may be configured to control generation and delivery of generated RF energy to the perineum or penis via at least one RF electrode or at least one return electrode, for example, to increase muscle elasticity.

In some embodiments, RF generator control circuitry may be configured to control an RF generator to generate RF energy having predetermined energy parameters. Predetermined energy parameters may include, for example, at least one of a predetermined frequency, phase, intensity, or polarity. In some embodiments, an RF generator or control circuitry may be configured to perform multiplexing of predetermined energy parameters to generate predetermined energy patterns. Predetermined energy patterns may include, for example, frequency patterns, phase patterns, intensity patterns, modulation patterns, or waveform pattern. In some embodiments, control circuitry may be configured to operate RF electrodes or RF electrode pairs according to a predetermined operation pattern. Predetermined operation patterns may include, for example, an intensity pattern, a mode pattern, a timing pattern, or a time duration of RF electrode operation.

In various embodiments, at least one of the energy parameters, energy patterns, operation patterns or any combination thereof may be determined and controlled based on a desired depth of RF energy delivery (e.g., depending on a target tissue) or based on a desired temperature value (e.g., depending on a desired treatment effect).

In some embodiments, RF electrodes need not be touching the skin, but rather may function via capacitance coupling through a film, or otherwise function through a thin layer or insulator between the electrode and the skin itself.

RF electrode and return electrode configurations may have a wide variety of arrangements to effectuate volumetric heating of urethra tissue, pelvic floor tissue, penis tissue, prostate gland tissue, or other bladder-proximal tissue. For example, in addition to the configurations shown in the instant figures, a unipolar configuration of RF electrode without return may be used, a return path through the ground may be used, a plate return electrode may be used at any position on the body, a bipolar or tripolar RF electrode configuration may be used, any arrangement of multipolar RF electrodes may be used, any arrangement of US transducers may be used, or any arrangement of a combination of RF electrodes and US transducers may be used.

Return electrodes may be positioned in a number of ways so as to induce current in the area of the pelvic floor when paired with one or more RF electrodes. In some embodiments, return electrodes may take the form of a plate electrode positioned on the penis some distance from perineal RF electrode(s), another position near the perineum, or positioned on any other part of the body, e.g., the foot.

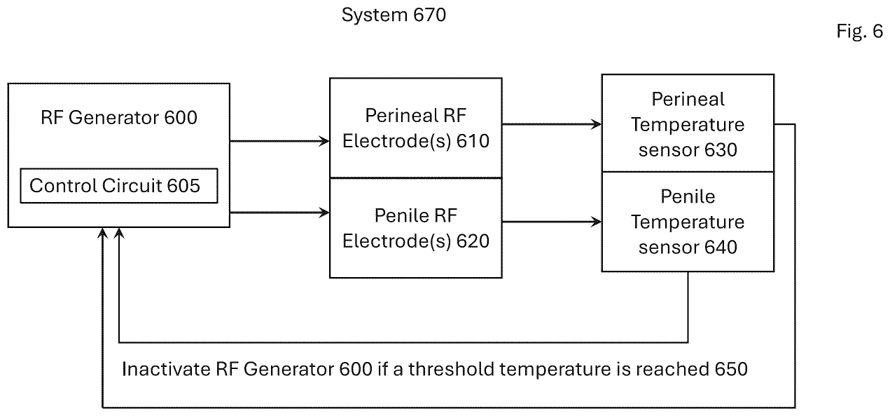

FIG. 6 illustrates a schematic drawing of a system for the treatment of urinary frequency disorders, including device components in an example embodiment of the instant application. FIG. 6 shows a basic circuit diagram showing control paths and feedback control for a system 670. Here, RF generator 600 or control circuit 605 signals to Perineal RF Electrode(s) 610 and Penile RF Electrode(s) 620 to initiate heating of the respective target tissues. In some embodiments, a synchronization circuit is present to synchronize application of RF pulses to the Perineal RF Electrode(s) 610 and Penile RF Electrode(s) 620 so that they are applied at the same time, in phase. In an alternative embodiment, the synchronization circuit is operable to shift the phase of the RF pulses to Perineal RF Electrode(s) 610 and Penile RF Electrode(s) 620 so that they are applied out of phase with each other.

In some embodiments, an arrangement of Penile RF Electrode(s) 620 may be affixed in a housing, e.g., made of plastic, to facilitate positioning on, around, or near the penis. In one embodiment the arrangement of Penile RF Electrode(s) 620 affixed in a housing may include a penile temperature sensor 640 positioned on the skin proximate to the target tissue for the detection of a temperature limit. Similarly, Perineal RF Electrode(s) 610 may be affixed within a pad or other housing structure to facilitate proper positioning of electrodes and temperature sensors, if any, on the perineum. In one embodiment, the pad or other housing structure may include a perineal temperature sensor 630 positioned on the skin proximate to the target tissue for the detection of a temperature limit.

In the above examples of temperature detection, temperature may be used as a signal to indicate desired heating of the target tissues. As a safety measure, a temperature threshold may be set to signal the RF generator 600 or control circuit 605 to cease operation, to prevent heat damage to skin or target tissues.

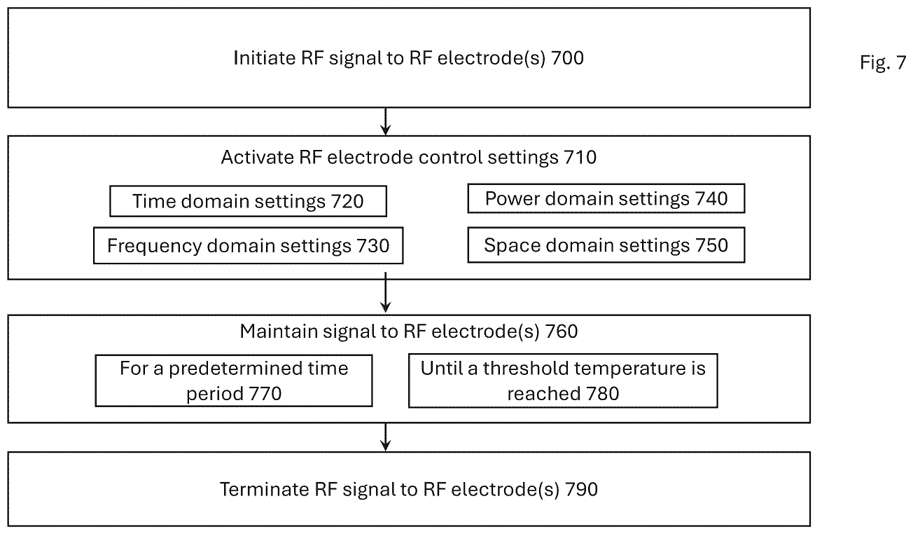

Accordingly, FIG. 7 describes examples of methods or software flow diagrams showing operations that may be carried out by system 670 or an ultrasound version of system 670.

The operations shown in FIG. 7 include initiating RF signal to RF electrode(s) 700, for example by a user activating an RF generator or control circuit. Next is an activation of RF electrode control settings 710, which may involve specification of time domain settings 720, frequency domain settings 730, power domain settings 740, or space domain settings 750. This may be followed by maintenance of the signal to RF electrode(s) 760 for a specified duration or series of pulses. This may be continued for a predetermined time period 770 or until a threshold temperature is reached 780. When either of those conditions is met, the RF generator or control circuit automatically terminates the RF signal to RF electrode(s) 790.

In some embodiments, a device is disclosed for the non-invasive treatment of urination frequency disorders, the device being applicable externally to a perineum portion of a patient (and optionally a penis portion), the device comprising a) at least one first ultrasound (US) transducer configured to generate US energy; wherein the at least one first ultrasound (US) transducer is configured for placement on a penile skin surface; b) at least one second ultrasound (US) transducer configured to generate US energy; wherein the at least one second ultrasound (US) transducer is configured for placement on a perineum skin surface; and c) at least one control circuit connected to the at least one first US transducer and the at least one second US transducer, wherein the at least one control circuit is operable to cause the first and second US transducers to transmit ultrasound energy through at least a portion of the penis and through at least a portion of pelvic floor tissue, respectively, to reduce urination frequency.

### Clauses

Clause 1. A method for treating abnormal urination frequency in a male patient, the method comprising:
a) applying energy non-invasively to a portion of the perineum, wherein the energy is operable to cause volumetric heating of one or more of a portion of a urethra, a portion of one or more pelvic floor muscles, a portion of a prostate gland, or a portion of a urinary bladder.

Clause 2. The method of clause 1, wherein the energy comprises at least one of radiofrequency energy or ultrasound energy applied to at least a portion of the skin of the perineum.

Clause 3. The method of clause 2, wherein the energy is applied across at least one of a time domain, a space domain, a frequency domain, or a power domain.

Clause 4. The method of clause 3, wherein the energy is applied statically.

Clause 5. The method of clause 3, wherein the energy is applied dynamically.

Clause 6. The method of clause 3, wherein the energy is varied across at least two domains.

Clause 7. The method of clause 1, wherein the energy is applied directionally relative to the anatomy of the pelvic floor.

Clause 8. The method of clause 7, wherein the energy is applied in one or more of the following modes:
back-to-front (posterior to anterior);
front-to-back (anterior to posterior);
bottom to top (inferior to superior);
top to bottom (superior to inferior);
left to right; or
right to left.

Clause 9. The method of clause 1, wherein the volumetric heating is operative to impact one or more portions of the ischiocavernosus muscle or the bulbospongiosus muscle.

Clause 10. The method of clause 1, wherein the applying energy non-invasively to a portion of the perineum comprises:
a) applying monopolar radiofrequency energy to the portion of the perineum.

Clause 11. The method of clause 10, wherein the applying monopolar radiofrequency energy to the portion of the perineum comprises:
a) applying radiofrequency energy via one electrode placed on the portion of the perineum, and a return plate placed elsewhere on the body of the male patient.

Clause 12. The method of clause 1, wherein the applying energy non-invasively to a portion of the perineum comprises:
a) applying bipolar radiofrequency energy to the portion of the perineum via one or more pairs of radiofrequency electrodes.

Clause 13. The method of clause 1, further comprising applying energy non-invasively to a portion of a penis, wherein the energy causes volumetric heating of the portion of the penis.

Clause 14. The method of clause 13, wherein the a) applying energy non-invasively to a portion of a penis; and b) applying energy non-invasively to a portion of the perineum are carried out in a coordinated manner.

Clause 15. The method of clause 14, wherein the coordinated manner comprises substantially alternating application of energy to the portion of the penis and to the portion of the perineum.

Clause 16. The method of clause 14, wherein the coordinated manner comprises substantially simultaneous application of energy to the portion of the penis and to the portion of the perineum.

Clause 17. The method of clause 1, comprising applying energy non-invasively to a portion of the perineum in a step-wise manner such that volumetric heating is effected at various depths beneath the skin of the portion of the perineum.

Clause 18. The method of clause 17, wherein the applying energy non-invasively to a portion of the perineum in a step-wise manner such that volumetric heating is effected at various depths beneath the skin of the portion of the perineum comprises:
i. applying radiofrequency energy non-invasively to a portion of the perineum via paired radiofrequency electrodes that are configured to direct radiofrequency energy to regions of pelvic floor muscles, urethra, prostate gland, or urinary bladder at various depths beneath the skin of the portion of the perineum.

Clause 19. The method of clause 1, wherein the volumetric heating occurs within a range of 0.5 mm to 15 mm below the portion of the perineum.

Clause 20. The method of clause 1, wherein the volumetric heating occurs for a period of time not to exceed three seconds.

Clause 21. The method of clause 1, wherein the volumetric heating is stopped before a target temperature of the one or more of a portion of a urethra, a portion of one or more pelvic floor muscles, a portion of a prostate gland, or a portion of a urinary bladder is reached.

Clause 22. The method of clause 1, further comprising:
a) applying radiofrequency energy to a target penile skin surface portion in coordination with the applying energy non-invasively to a portion of the perineum.

Clause 23. The method of clause 1, further comprising the administration of one or more of an anticholinergic medication, an alpha blocker medication, or botulinum toxin.

Clause 24. A method for treating abnormal urination frequency in a male patient, the method comprising:
a) applying energy non-invasively to a skin portion of the male patient, wherein the energy is operable to cause volumetric heating of one or more portions of the ischiocavernosus muscle or one or more portions of the bulbospongiosus muscle.

Clause 25. The method of clause 24 comprising:
a) applying energy non-invasively to a skin portion of the male patient, wherein the energy is operable to cause volumetric heating of one or more portions of the ischiocavernosus muscle proximate to its origin at the ischial tuberosity, or one or more portions of the bulbospongiosus muscle proximate to its origin at the perineal raphe.

Clause 26. A device for treating abnormal urination frequency in a male patient, the device comprising:
a) at least one energy emitting terminal configured to apply energy non-invasively to a portion of a penis, wherein the energy is operable to cause volumetric heating of the portion of the penis;
b) at least one energy emitting terminal configured to apply energy non-invasively to a portion of the perineum, wherein the energy is operable to cause volumetric heating of one or more of a portion of a urethra, a portion of one or more pelvic floor muscles, a portion of a prostate gland, or a portion of a urinary bladder; and
c) a controller configured to coordinate the operation of the energy emitting terminals of a) and b).

Clause 27. The device of clause 26, wherein the at least one energy emitting terminal configured to apply energy non-invasively to a portion of a penis comprises:
i. at least one radiofrequency (RF) electrode configured to apply RF energy to a skin portion of the penis.

Clause 28. The device of clause 26, wherein the at least one energy emitting terminal configured to apply energy non-invasively to a portion of a penis comprises:
i. at least one ultrasound (US) transducer configured to apply US energy to a skin portion of the penis.

Clause 29. The device of clause 26, further comprising at least one temperature sensor configured to measure temperature at one or more penile skin surfaces.

Clause 30. The device of clause 26, wherein a first RF electrode and a plurality of return RF electrodes are affixed to a housing such that they are positionable in a ring-shaped configuration around a circumference of the penis.

Clause 31. The device of clause 26, wherein a first RF electrode and a plurality of return electrodes are affixed to a housing such that they are positionable in a U-shaped configuration around a portion of the penis.

Clause 32. The device of clause 26, wherein a first RF electrode and a plurality of return electrodes are affixed to a housing such that they are positionable in a substantially linear configuration on a portion of the penis.

Clause 33. The device of clause 26, wherein a first RF electrode and a plurality of return electrodes are positioned in substantially any configuration on a portion of the penis.

Clause 34. The device of clause 33, wherein the at least one RF electrode comprises at least one of a microneedle electrode, a minimally invasive electrode, or a capacitance-coupled electrode.

Clause 35. The device of clause 26, wherein the at least one energy emitting terminal configured to apply energy non-invasively to a portion of the perineum comprises:
i. a two-dimensional pad including one or more pad RF electrodes, wherein the pad is configured to be applied to at least a portion of the perineum of the patient.

Clause 36. The device of clause 35, wherein the two-dimensional pad including one or more pad RF electrodes comprises:
i. an array of pad RF electrodes arranged in a grid pattern on the two-dimensional pad.

Clause 37. The device of clause 36, wherein the array of pad RF electrodes arranged in a grid pattern on the two-dimensional pad comprises:
i. an array of independently activatable pad RF electrodes configured for distributed or phased transmission of RF energy among the independently activatable pad RF electrodes;
   1. whereby skin in contact with the two-dimensional pad is maintained at or below a certain temperature level to avoid damaging the skin during substantially continuous delivery of RF energy to pelvic floor tissue over a period of time.

Clause 38. The device of clause 36, wherein the array of pad RF electrodes arranged in a grid pattern on the two-dimensional pad comprises:
i. an array of nine pad RF electrodes arranged in a three-by-three grid pattern on the two-dimensional pad.

Clause 39. The device of clause 26, further comprising at least one temperature sensor configured to measure temperature at one or more perineum skin surfaces.

Clause 40. A device operable carry out any of the methods of clauses 1-25 above.

Clause 41. A device for treating abnormal urination frequency in a male patient, the device comprising:
a) at least one radiofrequency (RF) generator configured to generate RF energy;
b) at least one RF electrode suitable for placement on a target perineal surface;
c) and at least one control circuit connected to the RF generator,
   i. wherein the at least one control circuit is operable to cause the RF energy to selectively heat at least one of a portion of a urethra, a portion of one or more pelvic floor muscles, a portion of a prostate gland, or a portion of a urinary bladder.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

In some implementations described herein, logic and similar implementations may include software or other control structures suitable to operation. Electronic circuitry, for example, may manifest one or more paths of electrical current constructed and arranged to implement various logic functions as described herein. In some implementations, one or more medias are configured to bear a device-detectable implementation if such media hold or transmit a special-purpose device instruction set operable to perform as described herein. In some variants, for example, this may manifest as an update or other modification of existing software or firmware, or of gate arrays or other programmable hardware, such as by performing a reception of or a transmission of one or more instructions in relation to one or more operations described herein. Alternatively, or additionally, in some variants, an implementation may include special-purpose hardware, software, firmware components, and/or general-purpose components executing or otherwise controlling special-purpose components. Specifications or other implementations may be transmitted by one or more instances of tangible or transitory transmission media as described herein, optionally by packet transmission or otherwise by passing through distributed media at various times.

Alternatively, or additionally, implementations may include executing a special-purpose instruction sequence or otherwise operating circuitry for enabling, triggering, coordinating, requesting, or otherwise causing one or more occurrences of any functional operations described above. In some variants, operational or other logical descriptions herein may be expressed directly as source code and compiled or otherwise expressed as an executable instruction sequence. In some contexts, for example, C++ or other code sequences can be compiled directly or otherwise implemented in high-level descriptor languages (e.g., a logic-synthesizable language, a hardware description language, a hardware design simulation, and/or other such similar modes of expression). Alternatively or additionally, some or all of the logical expression may be manifested as a Verilog-type hardware description or other circuitry model before physical implementation in hardware, especially for basic operations or timing-critical applications. Those skilled in the art will recognize how to obtain, configure, and optimize suitable transmission or computational elements, material supplies, actuators, or other common structures in light of these teachings.

Those having ordinary skill in the art will recognize that the state of the art has progressed to the point where there is little distinction left between hardware, software, and/or firmware implementations of aspects of systems; the use of hardware, software, and/or firmware is generally a design choice representing cost vs. efficiency tradeoffs (but not always, in that in certain contexts the choice between hardware and software can become significant). Those having ordinary skill in the art will appreciate that there are various vehicles by which processes and/or systems and/or other technologies described herein can be affected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be affected, none of which is inherently superior to the other in that any vehicle to be utilized is a choice dependent upon the context in which the vehicle will be deployed and the specific concerns (e.g., speed, flexibility, or predictability) of the implementer, any of which may vary.
The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those having ordinary skill in the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a USB drive, a solid state memory device, a hard disk drive, a Compact Disc (CD), a Digital Video Disk (DVD), a digital tape, a computer memory, etc. ; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).
In a general sense, those skilled in the art will recognize that the various aspects described herein which can be implemented, individually and/or collectively, by a wide range of hardware, software, firmware, and/or any combination thereof can be viewed as being composed of various types of “electrical circuitry.” Consequently, as used herein “electrical circuitry” or “control circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read-only, etc.)), and/or electrical circuitry forming a communications device (e.g., a modem, communications switch, optical-electrical equipment, etc.). Those having ordinary skill in the art will recognize that the subject matter described herein may be implemented in an analog or digital fashion or some combination thereof.
Those skilled in the art will recognize that at least a portion of the devices and/or processes described herein can be integrated into a data processing system. Those having ordinary skill in the art will recognize that a data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

In certain cases, use of a system or method as disclosed and claimed herein may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory).

A sale of a system or method may likewise occur in a territory even if components of the system or method are located and/or used outside the territory.

Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., operations), devices, objects, and the discussion accompanying them are used as examples for the sake of conceptual clarity and that various configuration modifications are contemplated. Consequently, as used herein, the specific examples set forth and the accompanying discussion are intended to be representative of their more general classes. In general, use of any specific example is intended to be representative of its class, and the non-inclusion of specific components (e.g., operations), devices, and objects should not be taken to be limiting.

With respect to the use of substantially any plural and/or singular terms herein, those having ordinary skill in the art can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are presented merely as examples, and that in fact many other architectures may be implemented which achieve the same functionality. In a conceptual sense, any arrangement of components to achieve the same functionality is effectively “associated” such that the desired functionality is achieved. Therefore, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of “operably couplable” include but are not limited to physically mateable or physically interacting components, wirelessly interactable components, wirelessly interacting components, logically interacting components, or logically interactable components.

In some instances, one or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components, inactive-state components, or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such a recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having ordinary skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having ordinary skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented as sequences of operations, it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for treating abnormal urination frequency in a male patient, the method comprising:
applying energy non-invasively to a portion of the perineum, wherein the energy is operable to cause volumetric heating of one or more of a portion of a urethra, a portion of one or more pelvic floor muscles, a portion of a prostate gland, or a portion of a urinary bladder.

2. The method of claim 1, wherein the energy comprises at least one of radiofrequency energy or ultrasound energy applied to at least a portion of the skin of the perineum.

3. The method of claim 2, wherein the energy is applied across at least one of a time domain, a space domain, a frequency domain, or a power domain.

4. The method of claim 3, wherein the energy is applied statically.

5. The method of claim 3, wherein the energy is applied dynamically.

6. The method of claim 3, wherein the energy is varied across at least two domains.

7. The method of claim 1, wherein the energy is applied directionally relative to the anatomy of the pelvic floor.

8. The method of claim 7, wherein the energy is applied in one or more of the following modes:
a) back-to-front (posterior to anterior);
b) front-to-back (anterior to posterior);
c) bottom to top (inferior to superior);
d) top to bottom (superior to inferior);
e) left to right; or
f) right to left.

9. The method of claim 1, wherein the volumetric heating is operative to impact one or more portions of the ischiocavernosus muscle or the bulbospongiosus muscle.

10. The method of claim 1, wherein the applying energy non-invasively to a portion of the perineum comprises:
applying monopolar radiofrequency energy to the portion of the perineum.

11. The method of claim 10, wherein the applying monopolar radiofrequency energy to the portion of the perineum comprises:
applying radiofrequency energy via one electrode placed on the portion of the perineum, and a return plate placed elsewhere on the body of the male patient.

12. The method of claim 1, wherein the applying energy non-invasively to a portion of the perineum comprises:
applying bipolar radiofrequency energy to the portion of the perineum via one or more pairs of radiofrequency electrodes.

13. The method of claim 1, further comprising applying energy non-invasively to a portion of a penis, wherein the energy causes volumetric heating of the portion of the penis.

14. The method of claim 13, wherein the a) applying energy non-invasively to a portion of a penis;
and b) the applying energy non-invasively to a portion of the perineum are carried out in a coordinated manner.

15. The method of claim 14, wherein the coordinated manner comprises substantially alternating application of energy to the portion of the penis and to the portion of the perineum.

16. The method of claim 14, wherein the coordinated manner comprises substantially simultaneous application of energy to the portion of the penis and to the portion of the perineum.

17. The method of claim 1, comprising applying energy non-invasively to a portion of the perineum in a step-wise manner such that volumetric heating is effected at various depths beneath the skin of the portion of the perineum.

18. The method of claim 17, wherein the applying energy non-invasively to a portion of the perineum in a step-wise manner such that volumetric heating is effected at various depths beneath the skin of the portion of the perineum comprises:
applying radiofrequency energy non-invasively to a portion of the perineum via paired radiofrequency electrodes that are configured to direct radiofrequency energy to regions of pelvic floor muscles, urethra, prostate gland, or urinary bladder at various depths beneath the skin of the portion of the perineum.

19. The method of claim 1, wherein the volumetric heating occurs within a range of 0.5 mm to 15 mm below the portion of the perineum.

20. The method of claim 1, wherein the volumetric heating occurs for a period of time not to exceed three seconds.

21. The method of claim 1, wherein the volumetric heating is stopped before a target temperature of the one or more of a portion of a urethra, a portion of one or more pelvic floor muscles, a portion of a prostate gland, or a portion of a urinary bladder is reached.

22. The method of claim 1, further comprising:
applying radiofrequency energy to a target penile skin surface portion in coordination with the applying energy non-invasively to a portion of the perineum.

23. The method of claim 1, further comprising the administration of one or more of an anticholinergic medication, an alpha blocker medication, or botulinum toxin.

24. A method for treating abnormal urination frequency in a male patient, the method comprising:
applying energy non-invasively to a skin portion of the male patient, wherein the energy is operable to cause volumetric heating of one or more portions of the ischiocavernosus muscle or one or more portions of the bulbospongiosus muscle.

25. The method of claim 24 comprising:
applying energy non-invasively to a skin portion of the male patient, wherein the energy is operable to cause volumetric heating of one or more portions of the ischiocavernosus muscle proximate to its origin at the ischial tuberosity, or one or more portions of the bulbospongiosus muscle proximate to its origin at the perineal raphe.

26. A device for treating abnormal urination frequency in a male patient, the device comprising:
a) at least one energy emitting terminal configured to apply energy non-invasively to a portion of a perineum, wherein the energy is operable to cause volumetric heating of one or more of a portion of a urethra, a portion of one or more pelvic floor muscles, a portion of a prostate gland, or a portion of a urinary bladder;
b) at least one energy emitting terminal configured to apply energy non-invasively to a portion of a penis, wherein the energy is operable to cause volumetric heating of the portion of the penis; and
c) a controller configured to coordinate the operation of the energy emitting terminals of a) and b).

27. The device of claim 26, wherein the at least one energy emitting terminal configured to apply energy non-invasively to a portion of a penis comprises:
at least one radiofrequency (RF) electrode configured to apply RF energy to a skin portion of the penis.

28. The device of claim 26, wherein the at least one energy emitting terminal configured to apply energy non-invasively to a portion of a penis comprises:
at least one ultrasound (US) transducer configured to apply US energy to a skin portion of the penis.

29. The device of claim 26, further comprising at least one temperature sensor configured to measure temperature at one or more penile skin surfaces.

30. The device of claim 26, wherein a first RF electrode and a plurality of return RF electrodes are affixed to a housing such that they are positionable in a ring-shaped configuration around a circumference of the penis.

31. The device of claim 26, wherein a first RF electrode and a plurality of return electrodes are affixed to a housing such that they are positionable in a U-shaped configuration around a portion of the penis.

32. The device of claim 26, wherein a first RF electrode and a plurality of return electrodes are affixed to a housing such that they are positionable in a substantially linear configuration on a portion of the penis.

33. The device of claim 26, wherein a first RF electrode and a plurality of return electrodes are positioned in substantially any configuration on a portion of the penis.

34. The device of claim 27, wherein the at least one RF electrode comprises at least one of a microneedle electrode, a minimally invasive electrode, or a capacitance-coupled electrode.

35. The device of claim 27, wherein the at least one energy emitting terminal configured to apply energy non-invasively to a portion of the perineum comprises:
a two-dimensional pad including one or more pad RF electrodes, wherein the pad is configured to be applied to at least a portion of the perineum of the patient.

36. The device of claim 35, wherein the two-dimensional pad including one or more pad RF electrodes comprises:
an array of pad RF electrodes arranged in a grid pattern on the two-dimensional pad.

37. The device of claim 36, wherein the array of pad RF electrodes arranged in a grid pattern on the two-dimensional pad comprises:
an array of independently activatable pad RF electrodes configured for distributed or phased transmission of RF energy among the independently activatable pad RF electrodes;
whereby skin in contact with the two-dimensional pad is maintained at or below a certain temperature level to avoid damaging the skin during substantially continuous delivery of RF energy to pelvic floor tissue over a period of time.

38. The device of claim 36, wherein the array of pad RF electrodes arranged in a grid pattern on the two-dimensional pad comprises:
an array of nine pad RF electrodes arranged in a three-by-three grid pattern on the two-dimensional pad.

39. The device of claim 26, further comprising at least one temperature sensor configured to measure temperature at one or more perineum skin surfaces.
